(19) United States
(12) Patent Application Publication
(10) Pub. No.: US 2015/0359740 A1
(43) Pub. Date: Dec. 17, 2015

(54) COMPOSITION AND METHOD FOR COMPOUNDED THERAPY

(71) Applicant: CMPD LICENSING, LLC, Conroe, TX (US)

(72) Inventor: Jay Richard Ray, II, Conroe, TX (US)

(21) Appl. No.: 14/836,455

(22) Filed: Aug. 26, 2015

Related U.S. Application Data
(63) Continuation-in-part of application No. 13/448,088, filed on Apr. 16, 2012.
Application No. 13/448,088 is a continuation of application No. 13/409,738, filed on Mar. 1, 2012, now abandoned.
Application No. 13/409,738 is a continuation-in-part of application No. 13/337,598, filed on Dec. 27, 2011, now abandoned.

(51) Int. Cl. A61K 9/06; A61K 31/167; A61K 47/20; A61K 31/196; A61K 31/137; A61K 31/192; A61K 9/00; A61K 31/195

(52) U.S. Cl. CPC A61K 9/06; A61K 9/0014; A61K 31/167; A61K 31/195; A61K 31/196; A61K 31/137; A61K 31/192; A61K 47/20

(57) ABSTRACT

A compounded transdermal cream may include gabapentin, diclofenac, cyclobenzaprine, or a combination thereof. The compounded transdermal cream may further include lidocaine in an amount between approximately 0.5% to approximately 5.0% by weight of the compounded transdermal cream and prilocaine in an amount between approximately 0.5% to approximately 5.0% by weight of the compounded transdermal cream.

### CROSS-REFERENCE TO RELATED APPLICATIONS

This patent application is a continuation in-part of co-pending U.S. patent application Ser. No. 13/448,088, entitled Composition and Method for Compounded Therapy, filed Apr. 16, 2012, which is a continuation of U.S. patent application Ser. No. 13/409,738, entitled Composition and Method for Compounded Therapy, filed Mar. 1, 2012, now abandoned, which is a continuation-in-part of U.S. patent application Ser. No. 13/337,598, entitled Composition and Method for Compounded Therapy, filed Dec. 27, 2011, now abandoned.

### FIELD OF THE INVENTION

The present application relates to compounded therapies. In particular, the present application relates to compositions for compounded therapy and methods of compounding medications.

### BACKGROUND

Transdermal creams are employed to deliver medication to the skin of a patient. Conventional compositions intended for topical administration include EMLA cream, a eutectic mixture of lidocaine and prilocaine in an emulsified topical cream, such as disclosed by U.S. Pat. Nos. 6,299,902 and 4,562,060, which are incorporated herein by reference in their entireties. However, conventional transdermal creams may include various drawbacks, such as addressing limited medical conditions, creating adverse side effects, and/or having limited shelf lives. Additionally, conventional methods of manufacturing transdermal creams may be inefficient and/or lack precision with the amount of active ingredients, or have other drawbacks.

### SUMMARY

In another aspect, the present embodiments may include the presence of dimethyl sulfoxide (DMSO) and/or Sterile Water for Irrigation. Alternatively, the transdermal cream of the present embodiments may be compounded to have no bulk ingredients in it. For instance, during the methods discussed herein, the DMSO may be removed and replaced with Sterile Water for Irrigation or other purified water. The transdermal cream may be essentially or entirely DMSO-free.

In one aspect, a compounded transdermal cream includes gabapentin, diclofenac, cyclobenzaprine, or a combination thereof. The compounded transdermal cream further includes lidocaine in an amount between approximately 0.5% to approximately 5.0% by weight of the compounded transdermal cream and prilocaine in an amount between approximately 0.5% to approximately 5.0% by weight of the compounded transdermal cream.

The compounded transdermal cream may include gabapentin present in an amount approximately 6% by weight of the compounded transdermal cream. Lidocaine and prilocaine may each present in an amount approximately 2.0% by weight of the compounded transdermal cream. In one embodiment, the compounded transdermal cream does not include DMSO. In another embodiment, the compounded transdermal cream does include DMSO. Diclofenac may be present in an amount approximately 5% by weight of the compounded transdermal cream. Lidocaine and prilocaine may each be present in an amount approximately 2.0% by weight of the compounded transdermal cream. Gabapentin may be present in an amount approximately 3% by weight of the compounded transdermal cream. Cyclobenzaprine may be present in an amount approximately 1% by weight of the compounded transdermal cream. Gabapentin may be present in an amount approximately 3% by weight of the compounded transdermal cream wherein the compounded transdermal cream does not include DMSO. Cyclobenzaprine is present in an amount approximately 1% by weight of the compounded transdermal cream and lidocaine and prilocaine may each be present in an amount approximately 2.0% by weight of the compounded transdermal cream.

In another aspect, a compounded transdermal cream includes ibuprofen in an amount approximately 5% by weight of the compounded transdermal cream; lidocaine in an amount between approximately 0.5% to approximately 5.0% by weight of the compounded transdermal cream; and prilocaine in an amount between approximately 0.5% to approximately 5.0% by weight of the compounded transdermal cream, wherein the compounded transdermal cream does not include DMSO. In one embodiment, lidocaine and prilocaine are each present in an amount approximately 2.0% by weight of the compounded transdermal cream.

The above-described and other features and advantages of the present disclosure will be appreciated and understood by those skilled in the art from the following detailed description, drawings, and appended claims.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 depicts an exemplary method of compounding; and

FIG. 2 depicts another exemplary method of compounding.

### DETAILED DESCRIPTION OF THE INVENTION

The present embodiments may relate to topically delivered compounded medications for treatment of various ailments, such as pain, osteoarthritis, epilepsy, inflammation, muscle fatigue, spasms, and/or other ailments. In one aspect, a transdermal cream for the effective administration of multiple medications simultaneously for one or more ailments may be provided. The transdermal cream may include low concentrations of lidocaine, prilocaine, meloxicam, lamotrigine and/or topiramate, and other active ingredients.

Alternatively, the transdermal cream may include a base having both lidocaine and prilocaine, and to which is added a fine powder of one or more medications. The medication in fine powder form may be generated from grinding up tablets of NSAIDs (Non-Steroidal Anti-Inflammatory Drugs), anticonvulsants, nerve depressants, muscle relaxants, NMDA (N-Methyl-D-aspartate) receptor antagonists, opiate or opioid agonists, antidepressants, and/or other active agents. The fine powder may allow for precise amounts of the active ingredients to be added to the base. The transdermal cream may exhibit excellent storage characteristics, and avoid separation and/or degradation of the active ingredients from the base for substantial lengths of time.

In one aspect, a transdermal cream may include lidocaine in an amount between approximately 0.5% and approximately 7.0% by weight of the transdermal cream; prilocaine in an amount between approximately 0.5% and approximately 7.0% by weight of the transdermal cream; meloxicam in an amount between approximately 0.01% and approximately 5.0% by weight of the transdermal cream; and lamotrigine and/or topiramate in an amount between approximately 0.5% and approximately 5.0% by weight of the transdermal cream. As a result, the transdermal cream may allow for the topical administration of lidocaine, prilocaine, meloxicam, and lamotrigine and/or topiramate simultaneously during use. In one embodiment, the transdermal cream may comprise approximately 2.0% by weight lidocaine and prilocaine, respectively; approximately 0.09% by weight meloxicam; and approximately 2.5% by weight either lamotrigine or topiramate.

In another aspect, a method of compounding one or more medications with a transdermal cream for the topical administration of a compounded therapy may be provided. The method may include grinding up one or more tablets of a NSAID, an anticonvulsant, a nerve depressant, a muscle relaxant, a NMDA receptor antagonist, antidepressant, and/or an opiate or opioid agonist into a fine powder of medication. The method may also include adding the fine powder of medication to a transdermal cream containing both lidocaine and prilocaine, the transdermal cream including both lidocaine and prilocaine in an amount of between approximately 0.5% and approximately 7.0% by weight of the transdermal cream. The method may include adding the fine powder of medication to the transdermal cream in a sufficient amount such that the transdermal cream includes the medication that is ground up in an amount of between approximately 0.01% and approximately 5.0% by final weight of the transdermal cream.

The fine powder may be a fine powder of compounded medication that includes two or more active ingredients. For example, the active ingredients may comprise a NSAID, such as meloxicam, and a nerve depressant or an anticonvulsant, such as lamotrigine and/or topiramate. In one embodiment, an amount of ground up compounded medication is added to the base such that the final composition of the transdermal cream after the fine powder of compounded medication is added is approximately 2.0% by weight lidocaine, approximately 2.0% by weight prilocaine, approximately 0.09% by weight meloxicam, and approximately 2.5% by weight either lamotrigine or topiramate.

### I. Compositions for Compounded Therapy

The present embodiments may relate to a compounded medication program. The compounded medication program may address several ailments simultaneously. In one aspect, the present embodiments may be intended to intended to minimize skin damage or irritation caused by the topical administration of various medications. Administering low doses or applying transdermal creams or gels with low concentrations of one or more active ingredients may minimize adverse side effects, such as side effects that develop with prolonged usage.

For instance, Stevens-Johnson Syndrome (SJS) and toxic epidermal necrolysis (TEN) are two forms of life-threatening skin conditions. SJS is a potentially deadly skin disease that usually results from a drug reaction. Drugs that have been linked to SJS include, but are not limited to: NSAIDs, allopurinol, phenytoin, carbamazepine, barbiturates, anticonvulsants, and sulfa antibiotics. However, almost any drug (prescription or over-the-counter) could potentially cause SJS if a severe enough allergy is present.

The onset of severe symptoms in drug related SJS may not appear for 1-2 weeks after first taking the drug causing the allergic reaction. Initial non-specific symptoms such as coughing, aching, headaches, fevers, vomiting, and diarrhea are commonly seen. These symptoms are usually followed by a red rash across the face and trunk of the body, later followed by blisters, and in some situations the nails and hair begin to fall out.

SJS is a very serious and potentially deadly condition and should be treated accordingly. Discontinuation of the medication and treatment of the “new infection” with a suitable antibiotic is the first step. In some situations, a patient is treated in a burn unit if necessary. However, compounded therapies may administer lower doses of active agents topically, and thus the effect of any adverse skin reaction may be lowered due to the lower doses of agent that the patient is allergic to.

In view of the foregoing, the present embodiments may include providing, within a base composition, several medications that address different ailments. The medications may be mixed in low concentrations to minimize any adverse reaction to the topical cream or gel containing the several medications.

The medications may be mixed with the base composition for topical administration to a patient. The medications may include one or more local anesthetics, such as lidocaine, prilocaine, or benzocaine; one or more NSAIDs, such as meloxicam; and one or more nerve depressants and/or anticonvulsants, such as gabapentin, topiramate, or lamotrigine. The medications may also include one or more muscle relaxants, such as baclofen or cyclobenzaprine; one or more NMDA receptor antagonists, such as ketamine; and/or one or opiate or opioid agonists, such as C2 or C3 opiate agonists, or tramadol.

### II. Meloxicam/Lamotrigine/Lidocaine/Prilocaine Compounded Medication

In one aspect, a transdermal cream or gel may include lidocaine, prilocaine, meloxicam, and lamotrigine. Lidocaine and prilocaine are amide-type local anesthetic agents. They may come in commercially available creams.

The amount of lidocaine and prilocaine in the transdermal cream may be approximately the same. The amount of lidocaine and prilocaine may each be between approximately 0.5% and approximately 5.0% of the total weight of the transdermal cream. Alternatively, the amount of lidocaine and prilocaine may each be between approximately 1.0% and approximately 4.0% of the total weight of the transdermal cream, or between approximately 1.5% and approximately 3.0% of the total weight of the transdermal cream. In one preferred embodiment, the amount of lidocaine and prilocaine may each be approximately 2.0% of the total weight of the final transdermal cream or gel.

Meloxicam is a NSAID that may provide pain relief, such as pain relief for osteoarthritis or rheumatoid arthritis. In one aspect, the amount of meloxicam in the transdermal cream or gel may be less than that of the other active ingredients.

The amount of meloxicam in the transdermal cream may be between approximately 0.01% and approximately 5.0% of the total weight of the transdermal cream, or between approximately 0.03% and approximately 3.0% of the total weight of the transdermal cream. Preferably, the amount of meloxicam may be between approximately 0.05% and approximately 0.15% of the total weight of the transdermal cream. In one preferred embodiment, the amount of meloxicam may be approximately 0.09% of the total weight of the transdermal cream or gel.

Lamotrigine may be characterized as an anticonvulsant. It may be used as an antiepileptic drug to treat epilepsy or bi-polar disorders. In one aspect, the amount of lamotrigine in the transdermal cream or gel may be more than the other active ingredients, such as lidocaine, prilocaine, meloxicam, and/or other active ingredients.

The amount of lamotrigine in the transdermal cream may be between approximately 0.5% and approximately 5.0% of the total weight of the transdermal cream, or between approximately 1.5% and approximately 3.5% of the total weight of the transdermal cream. Preferably, the amount of lamotrigine may be between approximately 2.0% and approximately 3.0% of the total weight of the transdermal cream. In one preferred embodiment, the amount of lamotrigine may be approximately 2.5% of the total weight of the transdermal cream or gel.

### III. Meloxicam/Topiramate/Lidocaine/Prilocaine Compounded Medication

In one aspect, a transdermal cream or gel may include lidocaine, prilocaine, meloxicam, and topiramate. The amounts of lidocaine, prilocaine, and meloxicam may be as stated above. Alternatively, other amounts of lidocaine, prilocaine, and meloxicam may be used.

Topiramate may be characterized as an antiepileptic drug used to treat epilepsy or migraines. In one aspect, the amount of topiramate in the transdermal cream or gel may be more than the other active ingredients, such as lidocaine, prilocaine, meloxicam, and/or other active ingredients.

The amount of topiramate in the transdermal cream may be between approximately 0.5% and approximately 5.0% of the total weight of the transdermal cream, or between approximately 1.5% and approximately 3.5% of the total weight of the transdermal cream. Preferably, the amount of topiramate may be between approximately 2.0% and approximately 3.0% of the total weight of the transdermal cream. In one preferred embodiment, the amount of topiramate may be approximately 2.5% of the total weight of the transdermal cream or gel.

### IV. Exemplary Method of Compounding

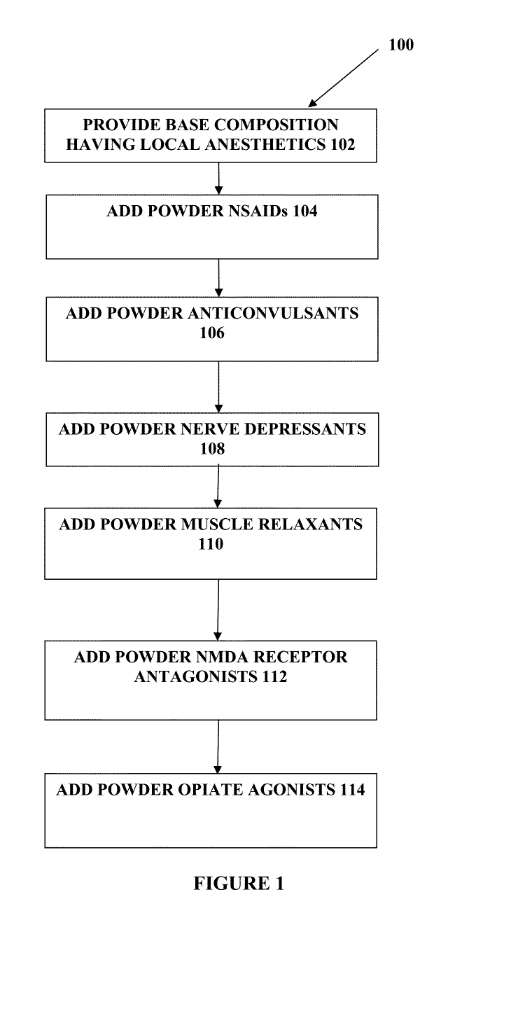

FIG. 1 depicts an exemplary method of compounding one or more medications with a transdermal cream or gel 100. The method 100 may include providing a base composition having one or more local anesthetics 102; and adding to the base a fine powder of medication comprising: one or more NSAIDs 104; one or more anticonvulsants 106; one or more or nerve depressants 108; one or more muscle relaxants 110; one or more NMDA receptor antagonists 112; and/or one or more opiate or opioid agonists 114. The transdermal cream or gel may include additional, fewer, or alternate steps and/or ingredients.

The method 100 may comprise providing a base composition 102. The base composition may comprise one or more local anesthetics 102. Primary examples of local anesthetics that the transdermal creams and base composition disclosed herein may employ include, but are not limited to, lidocaine, prilocaine, benzocaine, and/or tetracaine. The local anesthetics may comprise between approximately 0.1% and approximately 5.0% by weight of the transdermal cream. Other amounts may be used, including those discussed elsewhere herein. The base composition may include additional, fewer, or alternate ingredients.

Preferably, the base composition may include lidocaine and/or prilocaine. In one embodiment, the base composition may comprise an equal amount of lidocaine and prilocaine, such as between approximately 2.0% and approximately 3.0% by weight of the transdermal cream. Other amounts may be used, including those discussed elsewhere herein.

The method 100 may comprise adding to the base composition a fine powder of medication that includes one or more NSAIDs 104. NSAIDs may decrease inflammation, swelling, and pain. NSAIDs that may be added to the base composition may include: (1) oxicams—meloxicam and piroxicam; (2) salicylic acid derivatives—aspirin, diflunisal, salsalate, and trilisate; (3) propionic acids—flurbiprofen, ibuprofen, ketoprofen, naproxen, and oxaprozin; (4) acetic acids—diclofenac, etodolac, indomethacin, ketorolac, nabumetone, sulindac, and tolmetin; (5) fenamates—meclofenamate; and/or (6) COX-2 inhibitors—celecoxib, rofecoxib, and valdecoxib. Preferably, the final transdermal cream may comprise a low concentration of an oxicam, such as meloxicam or piroxicam, in a low amount between approximately 0.01% and 5.0% by weight of the final transdermal cream. In one embodiment, the final transdermal cream may include approximately 0.09% meloxicam by weight. Other amounts may be used, including those discussed elsewhere herein.

The method 100 may comprise adding to the base composition a fine powder of medication that includes one or more anticonvulsants 106. Anticonvulsants that may be added to the base composition may include lamotrigine and/or topiramate. The final transdermal cream may include an anticonvulsant in a low amount between approximately 0.1% and approximately 5.0% by weight of the final transdermal cream. Preferably, the final transdermal cream may comprise approximately 2.5% of either lamotrigine or topiramate by weight. Other amounts may be used, including those discussed elsewhere herein.

The method 100 may comprise adding to the base composition a fine powder of medication that includes one or more nerve depressants 108. Nerve depressants that may be added to the base composition may include gabapentin and/or others. The low amount of nerve depressant in the transdermal cream may be between approximately 0.1% and approximately 5.0% of the total weight of the transdermal cream. Other amounts may be used.

The method 100 may comprise adding to the base composition a fine powder of medication that includes one or more muscle relaxants 110. The active ingredients that may be added to the base compositions in form of fine powder may comprise baclofen, carisoprodol, chlorzoxazone, cyclobenzaprine, dantrolene, diazepam, metaxalone, methocarbamol, orphenadrine, quinine sulfate, tizanidine, and/or other muscle relaxants. The low amount of muscle relaxant in the transdermal cream may be between approximately 0.1% and approximately 5.0% of the total weight of the transdermal cream. Other amounts may be used.

The method 100 may comprise adding to the base composition a fine powder of medication that includes one or more NMDA receptor antagonists 112, such as ketamine. Ketamine may be useful because of its NMDA receptor activity (antagonism). The low amount of NMDA receptor antagonist in the transdermal cream may be between approximately 0.1% and approximately 5.0% of the total weight of the transdermal cream. Other amounts may be used.

The method 100 may comprise adding to the base composition a fine powder of medication that includes one or more opiate or opioid agonists 114. C2 opiate agonists may include oxycodone, morphine, methadone, hydromorphone, and fentanyl. C3 opiate agonists may include hydrocodone, codeine, propoxyphene, butalbital, and pentazocine. The active ingredients that may be added to the base composition in the form of fine powder may include the C2 and C3 opiate agonists named above and/or tramadol. The low amount of opiate or opioid agonist in the transdermal cream may be between approximately 0.1% and approximately 5.0% of the total weight of the transdermal cream. Other amounts may be used.

### V. Another Exemplary Method of Compounding

A method of compounding medications with a base composition using a fine powder of medication is disclosed herein. In general, a base composition, such as a lidocaine/prilocaine cream, should be selected. The preparer, such as a pharmacist, should calculate the weight of powders needed. Then, the prepare should grind the medication, such as tablets containing the medication, into fine powder and weigh the ingredients. The preparer should triturate the powders together and wet with dimethyl sulfoxide (DMSO) or Sterile Water for Irrigation. The preparer should bring to total weight with the lidocaine/prilocaine cream and mix well. The mixture should be milled in an ointment mill as necessary to acquire the desired consistency. After which, the preparer should mix thoroughly and package appropriately.

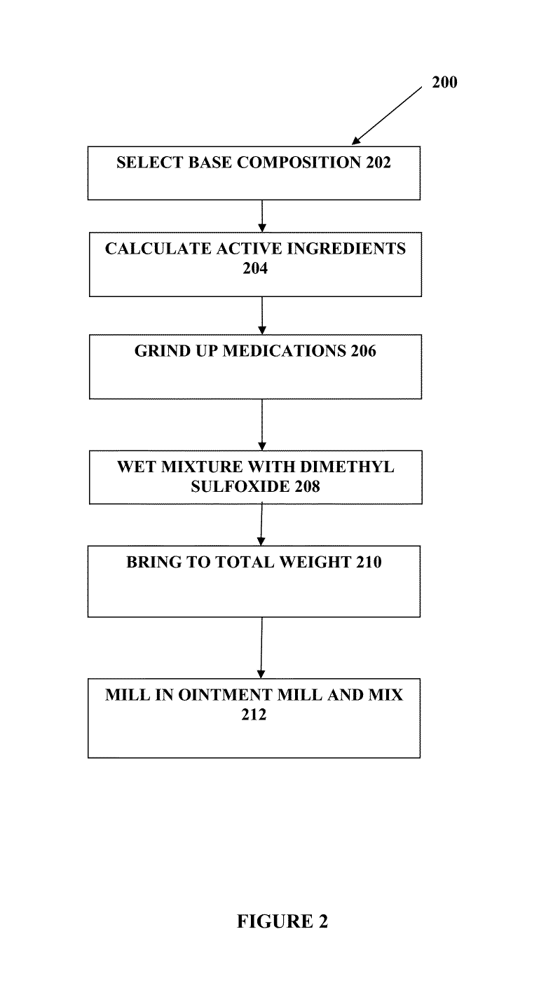

More specifically, FIG. 2 depicts an exemplary method of compounding medications with a transdermal cream 200. The method 200 depicted in FIG. 2 may be used to manufacture the transdermal creams discussed herein, including those discussed in relation to FIG. 1 above. The method 200 may include selecting a base composition 202; calculating an amount of active ingredients 204; grinding up the tablets containing the active ingredients 206; wetting the mixture with DMSO or Sterile Water for Irrigation 208; bringing to total weight 210; and milling in an ointment mill and mixing 212. The method 200 may include additional, fewer, or alternate actions.

The method 200 may include selecting a base composition 202 for a transdermal cream or gel. The base composition may include one or more local anesthetics, such as lidocaine and/or prilocaine. The base may include approximately equal amounts of lidocaine and prilocaine. The base composition may be a transdermal cream and may originally have approximately 2.5% lidocaine and approximately 2.5% prilocaine by weight. Other initial amounts of lidocaine and/or prilocaine may be used. In one embodiment, the base composition that includes lidocaine and/or prilocaine may be used in an amount of approximately 24,000 gm. Other amounts of base composition may be used.

The method 200 may include calculating an amount of active ingredients 204. The active ingredients may come in various size tablets. Noted herein, one of the transdermal cream embodiments, includes meloxicam and lamotrigine. For that embodiment, the ingredients may include 15 mg tablets of meloxicam, and approximately 1,500 of the 15 mg tables of meloxicam may be used. Tablets with other dosages of meloxicam may be used, and in different amounts. For instance, 7.5 mg or 30 mg tablets of meloxicam may be used.

The ingredients may also include 200 mg tablets of lamotrigine, and approximately 3,000 of the 200 mg tablets of lamotrigine may be used. Tablets with other dosages of lamotrigine may be used, and in different amounts. For instance, lamotrigine tablets ranging from 2 to 200 mg may be used.

To manufacture the transdermal cream embodiment that includes meloxicam and lamotrigine, the following formulas may be used to identify the amount of tablet powder of meloxicam and lamotrigine needed:

a. Meloxicam:

avg tab weight ______ gm×tablets needed ______=tablet powder needed ______ gm.

b. Lamotrigine:

avg tab weight ______ gm×tablets needed ______=tablet powder needed ______ gm.

The foregoing formulas may be used with the numbers stated above. For instance, the composition may require 1,500 of the 15 mg tables of meloxicam, and 3,000 of the 200 mg tablets of lamotrigine. As a result, in one embodiment, 22.5 grams of meloxicam and 600 grams of lamotrigine may be mixed with other ingredients, such as 24,000 gm of lidocaine 2.5%/prilocaine 2.5% cream, as well as 2,550 gm of dimethyl sulfoxide (DMSO). Instead of or in addition to lamotrigine, the medications added may include topiramate or other active ingredients. Instead of DMSO, Sterile Water for Irrigation may be used.

The method 200 may comprise grinding up the tablets containing the active ingredients 206. In one aspect, an automatic grinder may be used to grind up tablets containing one or more active ingredients into fine powder of medication. For instance, a Grindomix Mill may be used having a 100 volt, 60 Hz motor and five liter plastic container. The mill may have a standard lid, knife, and scraper. A five liter stainless steel container may be used that includes a knife holder. A knife of stainless steel may be used, and be autoclavable. The mill may have a plastic cover that is transparent.

The grinding up of the active ingredients into fine powder may allow for more precise amounts of each active ingredient in the final transdermal cream. This may be especially important when adding low amounts of active ingredients such that the final transdermal cream has low concentrations of various medications, which may reduce adverse allergic reactions to prolonged usage.

The method may include wetting the mixture with DMSO or Sterile Water for Irrigation 208. The DMSO and/or Sterile Water for Irrigation may facilitate the active ingredients penetrating the skin. After the ingredients in fine powder form are weighed, the preparer may triturate the powders of each ingredient together and wet with DMSO. For the 24,000 gm amount of lidocaine/prilocaine cream noted above, DMSO may be used in an amount of approximately 2,550 gm. Other amounts of DMSO may be used.

Instead of DMSO, the method may include wetting the mixture with only or primarily Sterile Water for Irrigation. Sterile Water for Irrigation USP may be a sterile, hypotonic, nonpyrogenic irrigating fluid or pharmaceutic aid (solvent), and may be composed of Sterile Water for Injection USP. It may be prepared by distillation and may contain no antimicrobial or bacteriostatic agents or added buffers. The pH may be about 5.7, or between 5.0 and 7.0. Sterile Water for Irrigation may be intended for use only as a single-dose, and may be classified as a sterile irrigant, wash, rinse, diluent and pharmaceutical vehicle. Instead of or addition to Sterile Water for Irrigation, Sterile Water for Injection or purified water may be used.

The method may include bringing to total weight with the lidocaine/prilocaine cream and mixing well 210. As noted elsewhere herein, after the fine powder of medication is mixed with the lidocaine/prilocaine base, the final transdermal cream may have approximately 2.0% by weight lidocaine, approximately 2.0% by weight prilocaine, approximately 0.09% by weight meloxicam, and approximately 2.5% by weight either lamotrigine or topiramate. The final transdermal cream may have other active ingredients as well, including those mentioned herein.

The method 200 may include milling the mixture in an ointment mill as necessary to acquire the desired consistency 212. After which, the preparer may mix the milled mixture thoroughly and package it in appropriate containers.

### VI. Exemplary Storage Characteristics

The compounded transdermal creams discussed herein that are made using fine powder of medication may exhibit excellent storage characteristics, and avoid separation and/or degradation of the active ingredients from a base composition for substantial lengths of time, such as six months or greater. For example, Table I below depicts the results of a 198 day potency test for a transdermal cream including meloxicam, lamotrigine, lidocaine, and prilocaine. As shown, there is little degradation of the active ingredients. The sample was stored in approximately 20° C. to 25° C. (68° F. to 77° F.) conditions, and contained one large white tube with cream in a clear bag.

TABLE I
198 Day Potency Test

| | Expected | | | % | Test |
|---|---|---|---|---|---|
| Analyte/Specifications | Amount | Units | Results | of EXP. | Method |
| Lamotrigine | 2.5 | % | 2.463 | 98.5% | HPLC |
| Specifications = N/A | | | | | |
| Lidocaine | 2.0 | % | 1.927 | 96.4% | HPLC |
| Specifications = N/A | | | | | |
| Meloxicam | 0.09 | % | 0.0962 | 106.9% | HPLC |
| Specifications = N/A | | | | | |
| Prilocaine | 2.0 | % | 2.118 | 105.9% | HPLC |
| Specifications = N/A | | | | | |

Table II below depicts the results of a 100 day potency test for a transdermal cream including meloxicam, topiramate, lidocaine, and prilocaine. As shown, there is little degradation of the active ingredients. The sample was stored in approximately 20° C. to 25° C. (68° F. to 77° F.) conditions, and contained one large white tube with cream in a clear bag.

TABLE II
100 Day Potency Test

| | Expected | | | % | Test |
|---|---|---|---|---|---|
| Analyte/Specifications | Amount | Units | Results | of EXP. | Method |
| Lidocaine | 2.0 | % | 1.700 | 85.0% | HPLC |
| Specifications = N/A | | | | | |
| Meloxicam | 0.09 | % | 0.0945 | 105.0% | HPLC |
| Specifications = N/A | | | | | |
| Prilocaine | 2.0 | % | 1.899 | 95.0% | HPLC |
| Specifications = N/A | | | | | |
| Topiramate | 2.5 | % | 2.368 | 94.7% | HPLC |
| Specifications = N/A | | | | | |

### VII. Exemplary Methods of Compounding Using Fine Powder

An exemplary method of compounding may include grinding up tablets of one or more active ingredients into a fine powder, and then adding those ingredients in powder form to a compounded transdermal cream or gel. The active ingredients that are ground up into a fine powder of medication may include one or more NSAIDs, anticonvulsants, nerve depressants, muscle relaxants, antidepressants, NMDA receptor antagonists, opioid or opiate agonists, local anesthetics, and/or other active agents. The transdermal cream or gel may or may not have one or more pre-existing ingredients prior to the addition of the fine powder of medication, such as one or more pre-existing local anesthetics.

The method may include grinding up tablets of one or more local anesthetics into a fine powder. The local anesthetics ground up into powder form may include lidocaine and/or prilocaine, or other agents. An amount of lidocaine and/or prilocaine powder may be added to the transdermal cream such that lidocaine comprises between approximately 0.5% and approximately 7.0% by weight of the transdermal cream, and that prilocaine comprises between approximately 0.5% and approximately 7.0% by weight of the transdermal cream. Other amounts may be used, including those discussed elsewhere herein.

The method may include grinding up tablets of one or more NSAIDs into a fine powder of medication. The NSAIDs that are ground up may include meloxicam, fluribiprofen, nabumetone, and/or other NSAIDs. The amount of NSAIDs may be between approximately 0.05% and 25.0% by weight of the transdermal cream. For instance, the transdermal cream may include meloxicam in a low amount of between approximately 0.05% and approximately 0.15% by weight of the transdermal cream, and/or flurbiprofen or nabumetone in an amount between approximately 5.0% and approximately 25.0% of the transdermal cream by weight. Other amounts may be used, including those discussed elsewhere herein.

The method may include grinding up tablets of one or more anticonvulsants into the fine powder of medication. The anticonvulsants that are ground up may include lamotrigine, topiramate, and/or other anticonvulsants. The transdermal cream may include an amount of anticonvulsant of between approximately 1.0% and approximately 5.0% by weight of the transdermal cream. Other amounts may be used, including those discussed elsewhere herein.

The method may include grinding up tablets of one or more muscle relaxants into a fine powder of medication. The muscle relaxants that are ground up may include baclofen, cyclobenzaprine, and/or other muscle relaxants. The transdermal cream may include an amount of muscle relaxant of between approximately 1.0% and approximately 5.0% by weight of the transdermal cream. Other amounts may be used, including those discussed elsewhere herein.

The method may include grinding up tablets of one or more opioid or opiate agonists into a fine powder of medication. The opioid or opiate agonists that are ground up may include C2 or C3 opiate agonists, tramadol, and/or others. The transdermal cream may include an amount of opioid or opiate agonist of between approximately 1.0% and approximately 5.0% by weight of the transdermal cream. Other amounts may be used, including those discussed elsewhere herein.

The method may include grinding up tablets of one or more NMDA receptor antagonists into a fine powder of medication. The NMDA receptor antagonists that are ground up may be ketamine and/or other antagonists. The transdermal cream may include an amount of NMDA receptor antagonist of between approximately 1.0% and approximately 40.0% by weight of the transdermal cream. Other amounts may be used, including those discussed elsewhere herein.

The method may include grinding up tablets of one or more nerve depressants into a fine powder of medication. The nerve depressants that are ground up may include gabapentin and/or other nerve depressants. The transdermal cream may include an amount of nerve depressant of between approximately 1.0% and approximately 15.0% by weight of the transdermal cream. Other amounts may be used, including those discussed elsewhere herein.

The method may include grinding up tablets of one or more tricyclic antidepressants or other antidepressants into a fine powder of medication. The tricyclic antidepressants that are ground up may include amitriptyline and/or other antidepressants. The transdermal cream may include an amount of antidepressant of between approximately 1.0% and approximately 15.0% by weight of the transdermal cream. Other amounts may be used, including those discussed elsewhere herein.

The fine powder of each active ingredient that is ground up may be added to a transdermal cream or gel separately or collectively. The medications may comprise approximately 20%, approximately 30%, or approximately 40% or more of a transdermal cream by weight. Other amounts may be used, including those discussed elsewhere herein. Alternatively, administering low doses or applying transdermal creams or gels with low concentrations of one or more active ingredients may minimize adverse side effects, such as adverse skin conditions that may develop with usage. Therefore, the method may include adding several medications in fine powder form to a transdermal cream or gel to alleviate the magnitude of any adverse skin conditions that may arise, while simultaneously providing a compounded therapy.

In specific embodiments, the two or more medications that are ground up into a fine powder may include (1) a NSAID (such as meloxicam) and an anticonvulsant (such as lamotrigine and/or topiramate); (2) a NSAID (such as fluribiprofen or nabumetone), a nerve depressant (such as gabapentin), and a muscle relaxant (such as baclofen or cyclobenzaprine); or (3) a NSAID (such as fluribiprofen or nabumetone), a nerve depressant (such as gabapentin), and an antidepressant (such as amitriptyline). Other combinations of medications may be used.

In one aspect, an amount of fine powder of several medications may be ground up and then added to a transdermal cream or gel. The several medications may include: (1) at least one local anesthetic, such as lidocaine and/or prilocaine, in an amount between approximately 1.0% and approximately 7.0% of the transdermal cream by weight; (2) at least one nerve depressant, such as gabapentin, in an amount between approximately 5.0% and approximately 15.0% of the transdermal cream by weight; (3) at least one NSAID, such as flurbiprofen or nabumetone, in an amount between approximately 5.0% and approximately 25.0% of the transdermal cream by weight; and/or (4) at least one muscle relaxant, such cyclobenzaprine, in an amount between approximately 0.5% and approximately 4.0% of the transdermal cream by weight such that multiple ailments may be addressed simultaneously. In one embodiment, the transdermal cream may comprise, by weight of the transdermal cream, approximately 2.0% lidocaine, approximately 2.0% prilocaine, approximately 6.0% gabapentin, approximately 1.0% cyclobenzaprine, and approximately 10.0% flurbiprofen or approximately 20% nabumetone. The several medications may also include an opioid or opiate agonist, a tricyclic or other antidepressant, a NMDA receptor antagonist, and/or other active ingredients.

In another aspect, an amount of fine powder of several medications may be ground up and then added to a transdermal cream or gel. The several medications may include: (1) at least one local anesthetic, such as lidocaine and/or prilocaine, in an amount between approximately 1.0% and approximately 7.0% of the transdermal cream by weight; (2) at least one nerve depressant, such as gabapentin, in an amount between approximately 5.0% and approximately 15.0% of the transdermal cream by weight; (3) at least one NSAID, such as flurbiprofen or nabumetone, in an amount between approximately 5.0% and approximately 25.0% of the transdermal cream by weight; and/or (4) at least one tricyclic antidepressant, such as amitriptyline, in an amount between approximately 0.5% and approximately 4.0% of the transdermal cream by weight. In one embodiment, the transdermal cream may comprise, by weight of the transdermal cream, approximately 2.0% lidocaine, approximately 2.0% prilocaine, approximately 6.0% gabapentin, approximately 1.0% amitriptyline, and approximately 10.0% flurbiprofen or approximately 20.0% nabumetone. The several medications may also include an opioid or opiate agonist, a muscle relaxant, a NMDA receptor antagonist, and/or other active ingredients.

In another aspect, an amount of fine powder of several medications may be ground up and then added to a transdermal cream or gel. The transdermal cream may include lidocaine in an amount between approximately 0.5% and approximately 7.0% by weight of the transdermal cream; prilocaine in an amount between approximately 0.5% and approximately 7.0% by weight of the transdermal cream; meloxicam in an amount between approximately 0.01% and approximately 5.0% by weight of the transdermal cream; and lamotrigine and/or topiramate in an amount between approximately 0.5% and approximately 5.0% by weight of the transdermal cream. In one embodiment, the transdermal cream may comprise approximately 2.0% by weight of both lidocaine and prilocaine, approximately 0.09% by weight meloxicam, and approximately 2.5% by weight lamotrigine and/or topiramate. As a result, the transdermal cream or gel may allow for the topical administration of lidocaine, prilocaine, meloxicam, and lamotrigine and/or topiramate simultaneously during use. The several medications may also include an opioid or opiate agonist, a muscle relaxant, a NMDA receptor antagonist, a nerve depressant, other NSAIDs, other anticonvulsants, and/or other active agents, including those discussed elsewhere herein.

In another aspect, the transdermal cream comprises a nerve depressant, lidocaine, and prilocaine. A fine powder medication of one or more of the above medications may be obtained by crushing tablets of the medication, such as commercial tablets of the nerve depressant. In one such embodiment, the nerve depressant comprises or consists of gabapentin. In a further embodiment, the transdermal cream includes approximately 1% to approximately 10%, approximately 3% to approximately 9%, or approximately 5% to approximately 8% by weight gabapentin and approximately 0.5% to approximately 5.0% by weight of each of lidocaine and prilocaine with DMSO or without DMSO. In one such embodiment, the transdermal cream includes approximately 6% by weight gabapentin and approximately 2% by weight of each of lidocaine and prilocaine. In one embodiment of the above transdermal cream, the transdermal cream includes DMSO. In another embodiment, the transdermal cream does not include DMSO, e.g., DMSO-free.

In a further aspect, the transdermal cream may comprise a nerve depressant, lidocaine, prilocaine, and a NSAID. A fine powder medication of one or more of the above medications may be obtained by crushing tablets of the medication, such as commercial tablets of the nerve depressant and the NSAID. The fine powder medication may also be obtained from bulk sources, which may include powder medication that may be subsequently ground to fine powder or be provided in a fine powder form. In one particular instance of the above embodiment, the nerve depressant comprises or consists of gabapentin and the NSAID comprises or consists of diclofenac. In one embodiment, gabapentin and diclofenac are present in the transdermal cream in an amount approximately 1% to approximately 10%, approximately 1% to approximately 6%, or approximately 2% to approximately 5% by weight gabapentin, approximately 1% to approximately 10%, approximately 2% to approximately 8%, approximately 3% to approximately 7%, or approximately 4% to approximately 6% by weight diclofenac, and approximately 0.5% to approximately 5.0% by weight of each of lidocaine and prilocaine with DMSO or without DMSO. In one such embodiment, the transdermal cream includes approximately 3% by weight gabapentin, approximately 5% by weight diclofenac, and approximately 2% by weight of each of lidocaine and prilocaine. In one embodiment the transdermal cream includes DMSO. In another embodiment the transdermal cream does not include DMSO, e.g., DMSO-free.

In a further aspect, the transdermal cream may comprise a nerve depressant, an NSAID, lidocaine, prilocaine, and a muscle relaxant. A fine powder medication of one or more of the above active ingredients may be obtained by crushing tablets of the medication, such as commercial tablets of the nerve depressant, the NSAID, and the muscle relaxant. The fine powder medication may also be obtained from bulk sources, which may include powder medication that may be subsequently ground to fine powder or be provided in a fine powder form. In one particular instance of the above embodiment, the nerve depressant comprises or consists of gabapentin, the NSAID comprises or consists of diclofenac, and the muscle relaxant comprises cyclobenzaprine. In one embodiment, gabapentin, diclofenac, and cyclobenzaprine are present in the transdermal cream in an amount approximately 1% to approximately 10%, approximately 1% to approximately 6%, or approximately 2% to approximately 5% by weight gabapentin, approximately 1% to approximately 10%, approximately 2% to approximately 8%, approximately 3% to approximately 7%, or approximately 4% to approximately 6% by weight diclofenac, approximately 0.5% to approximately 2%, approximately 0.5% to approximately 1.5% by weight cyclobenzaprine, and approximately 0.5% to approximately 5.0% by weight of each of lidocaine and prilocaine with DMSO or without DMSO. In one such embodiment, the compounded transdermal cream includes approximately 3% by weight gabapentin, approximately 5% by weight diclofenac, approximately 1% by weight cyclobenzaprine, and approximately 2% by weight of each of lidocaine and prilocaine. In one embodiment the compounded transdermal cream includes DMSO. In another embodiment the compounded transdermal cream does not include DMSO, e.g., DMSO-free.

In one aspect, the compounded transdermal cream comprises a NSAID, lidocaine, and prilocaine. A fine powder medication of one or more of the above medications may be obtained by crushing tablets of the medication, such as commercial tablets of the NSAID. In one such embodiment, the NSAID is diclofenac. In a further embodiment, the compounded transdermal cream includes approximately 1% to approximately 10%, approximately 2% to approximately 8%, or approximately 4% to approximately 6% by weight of diclofenac and approximately 0.5% to approximately 5.0% by weight of each of lidocaine and prilocaine with DMSO or without DMSO. In one such embodiment, the compounded transdermal cream includes approximately 5% by weight of diclofenac and approximately 2% by weight of each of lidocaine and prilocaine. In one embodiment of the above transdermal cream, the transdermal cream comprising diclofenac includes DMSO. In another embodiment, the transdermal cream comprising diclofenac does not include DMSO, e.g., DMSO-free.

### VIII. Exemplary Embodiments Methods of Compounding Using Solution

In one method of formulating a topically delivered compounded medication, one or more of the active ingredients are provided in an aqueous solution and combined with the base composition comprising lidocaine and prilocaine cream. The lidocaine and prilocaine cream preferably comprises an eutectic mixture of equal quantities (by weight) of lidocaine and prilocaine. The lidocaine and prilocaine cream may thus include an emulsifier. The lidocaine and prilocaine cream may further comprise lidocaine and prilocaine in an emulsion preparation wherein lidocaine and prilocaine are provided at a 1:1 ratio. Preferably the oil phase of the emulsion preparation comprises an eutectic mixture of lidocaine and prilocaine in a ratio of 1:1 by weight. For example, in one embodiment, the lidocaine and prilocaine cream comprises a 5% emulsion preparation, containing 2.5% each of lidocaine and prilocaine. In one embodiment, the lidocaine and prilocaine cream comprises an emulsifier comprising polyoxyethylene fatty acid esters. The lidocaine and prilocaine cream may further comprise a thickening agent. In one embodiment, the thickening agent comprises carboxypolymethylene. The lidocaine and prilocaine cream may further comprise additional excipients or inactive components such as sodium hydroxide and purified water.

In one embodiment, the method of formulating a topically delivered medication in which one or more active ingredients are provided in an aqueous solution and then combined and mixed with the base composition includes combining the aqueous solution and the base composition, wherein lidocaine and prilocaine are already in the cream, such as premixed or pre-incorporated into the cream. For example, the base composition may be a commercially manufactured lidocaine and prilocaine cream, such as lidocaine 2.5% and prilocaine 2.5% cream. In some such embodiments, a suitable lidocaine and prilocaine cream may be a lidocaine and prilocaine cream marketed under the trade name EMLA (Eutectic Mixture of Local Anesthetics) or a generic lidocaine and prilocaine cream, e.g., a lidocaine and prilocaine cream such as those manufactured by Hi-Tech Pharmacal Co., Inc., Amityville, N.Y., or E. Fougera & Co., a division of Fougera Pharmaceuticals Inc., Melville, N.Y. The above commercially manufactured lidocaine and prilocaine creams comprise a 5% emulsion preparation, containing approximately 2.5% of each of lidocaine and prilocaine. The lidocaine and prilocaine cream is provided in an emulsion in which the oil phase is a eutectic mixture of lidocaine and prilocaine present in a ratio of 1:1 by weight, having a melting point below room temperature, and, therefore, both local anesthetics exist as a liquid oil rather than as crystals at room temperature. Each gram of the lidocaine and prilocaine cream may contain lidocaine in an amount approximately 25 mg, prilocaine in an amount approximately 25 mg, polyoxyethylene fatty acid esters (as emulsifiers), carboxypolymethylene or carbomer 934 (as a thickening agent), sodium hydroxide, and purified water to 1 gram.

In various embodiments, the at least one active ingredient in aqueous solution may comprise an NSAID. As described above, the NSAID combined with the base composition may include one or more of: (1) oxicams—meloxicam and piroxicam; (2) salicylic acid derivatives—aspirin, diflunisal, salsalate, and trilisate; (3) propionic acids—flurbiprofen, ibuprofen, ketoprofen, naproxen, and oxaprozin; (4) acetic acids—diclofenac, etodolac, indomethacin, ketorolac, nabumetone, sulindac, and tolmetin; (5) fenamates—meclofenamate; and/or (6) COX-2 inhibitors—celecoxib, rofecoxib, and valdecoxib. In one embodiment, the NSAID comprises a benzeneacetic acid derivative such as diclofenac or pharmaceutically acceptable salt thereof provided in an aqueous solution. For example, the diclofenac may be provided in an aqueous solution comprising a diclofenac sodium solution. In one embodiment, the diclofenac or pharmaceutically acceptable salt thereof may comprise a diclofenac sodium solution for topical application. The diclofenac sodium solution may contain, for example, 1.5% (w/w) diclofenac sodium wherein each 1 mL of solution may contain approximately 16.05 mg of diclofenac sodium. In one embodiment, the diclofenac solution comprises a diclofenac sodium solution 1.5% (w/w) such as that manufactured under the trade name PENNSAID® by Nuvo Manufacturing, Varennes, Quebec, Canada for treating the pain of osteoarthritis of the knee. The diclofenac solution may also contain various inactive ingredients such as dimethyl sulfoxide USP (DMSO, 45.5% w/w), ethanol, glycerin, propylene glycol and purified water. In one embodiment, the diclofenac solution comprises a diclofenac sodium solution marketed under the trade name PENNSAID® and manufactured by Nuvo Manufacturing, Varennes, Quebec, Canada, in a 2% (w/w) diclofenac solution for treating the pain of osteoarthritis of the knee. Each gram of solution may contain approximately 20 mg of diclofenac sodium and various inactive ingredients such as dimethyl sulfoxide USP (DMSO, 45.5% w/w), ethanol, purified water, propylene glycol, and hydroxypropyl cellulose. In other embodiments, other concentrations of diclofenac solution, such as diclofenac sodium solutions, may be used.

The compounded transdermal cream formulated by combining a commercial NSAID solution such as diclofenac sodium solution with lidocaine and prilocaine cream according to the embodiments described herein possess surprising stability. For example, formulations wherein components comprise a solution and a carboxy polymer cream base often times will “crack”. The compounded transdermal cream, however, has been found to be incredibly stable and pristine in appearance. In various embodiments, DMSO makes up approximately 45.5% of the diclofenac solution (1.5% Stock Solution) and comprises approximately 10% of the final finished compound, and the final compound may have approximately 5% DMSO in it.

The combined diclofenac solution and lidocaine and prilocaine cream may be milled, e.g., in an ointment mill, and blended to achieve a desired creamy consistency wherein the active ingredients are approximately evenly dispersed within the compounded transdermal cream.

The compounded transdermal cream formulated by combining a commercially manufactured lidocaine and prilocaine cream and a commercially manufactured diclofenac sodium solution such as a diclofenac sodium may comprise relatively low concentrations of the active ingredients compared to conventional topical formulations including one or more of the active ingredients. Due to the formulation and combination described herein, the present compounded transdermal cream may provide similar effectiveness while having an increased safety profile. The increased safety profile may be especially beneficial to patients with gastric bleeds, on blood thinners, etc. The compounded composition may also provide local anesthetics benefits while promoting deeper penetration into the skin and leveraging DMSO in the diclofenac sodium solution that would be embedded into the compounded transdermal cream.

In a one embodiment, a method of formulating a compounded medication product comprises combining a commercially manufactured lidocaine 2.5% and prilocaine 2.5% cream and a commercially manufactured diclofenac sodium solution such as a diclofenac sodium 1.5% (w/w) or diclofenac sodium 2.0% (w/w) solution to form a compounded transdermal cream whereby the final concentration by weight of the compounded transdermal cream comprises diclofenac or diclofenac sodium at a concentration of approximately 0.1% to approximately 1.0%, lidocaine at a concentration of approximately 1.5% to approximately 2.25%, and prilocaine at a concentration of approximately 1.5% to approximately 2.25%. In further embodiments, the method of formulating a compounded drug product comprises combining a commercially available lidocaine 2.5% and prilocaine 2.5% cream and a commercially available diclofenac solution such as a diclofenac sodium 1.5% (w/w) or diclofenac sodium 2.0% (w/w) solution whereby the final concentration by weight of the compounded drug product comprises diclofenac or diclofenac sodium at a concentration of approximately 0.1% to approximately 0.1% to approximately 0.75%, approximately 0.1% to approximately 0.5%, approximately 0.1% to approximately 0.3%, approximately 0.2% to approximately 0.75%, approximately 0.2% to approximately 0.5%, approximately 0.2% to approximately 0.3%, approximately 0.3% to approximately 0.75%, approximately 0.3% to approximately 0.5%, or approximately 0.5% to approximately 0.75%, lidocaine at a concentration of approximately 1.5% to approximately 2.25%, and prilocaine at a concentration of approximately 1.5% to approximately 2.25%. In further embodiments, the method may also include combining one or more additional active ingredients comprising one or more additional NSAIDs, one or more additional local anesthetics, one or more anticonvulsants, one or more nerve depressants, one or more muscle relaxants, one or more antidepressants, one or more NMDA receptor antagonists, or one or more opioid or opiate agonists, and/or other active agents.

The one or more additional NSAIDs that may be further added to or included in the compounded transdermal cream, e.g., combined with the lidocaine and prilocaine cream or the diclofenac sodium solution prior to combining the lidocaine and prilocaine cream and the diclofenac sodium solution or to the compounded transdermal cream comprising the combined diclofenac sodium solution and the lidocaine and prilocaine cream, may be present in an amount between approximately 0.1% and approximately 5.0% by weight of the final compounded transdermal cream and selected from salicylic acid derivatives selected from aspirin, diflunisal, salsalate, and trilisate; propionic acids selected from flurbiprofen, ibuprofen, ketoprofen, naproxen, and oxaprozin; tolmetin; eclofenamate; COX-2 inhibitors selected from celecoxib, rofecoxib, and valdecoxib, oxicams selected from meloxicam, piroxicam; or an additional acetic acid selected from etodolac, indomethacin, ketorolac, nabumetone, and sulindac. The one or more anticonvulsants selected from lamotrigine or topiramate may be added to or included in the compounded transdermal cream, e.g., combined with the lidocaine and prilocaine cream or the diclofenac sodium solution prior to combining the lidocaine and prilocaine cream and the diclofenac sodium solution or to the compounded transdermal cream comprising the combined diclofenac sodium solution and the lidocaine and prilocaine cream, in an amount between approximately 0.1% and approximately 5.0% of the total weight of the final compounded transdermal cream. The one or more nerve depressants may include gabapentin added to or included in the compounded transdermal cream comprising diclofenac, lidocaine, and prilocaine in an amount between approximately 0.1% and approximately 5.0% of the total weight of the final compounded transdermal cream. The one or more muscle relaxants that may be added to or included in the compounded transdermal cream comprising diclofenac, lidocaine, and prilocaine may be provided in an amount between approximately 0.1% and approximately 5.0% of the total weight of the final compounded transdermal cream, wherein the one or more muscle relaxants are selected from baclofen, carisoprodol, chlorzoxazone, cyclobenzaprine, dantrolene, diazepam, metaxalone, methocarbamol, orphenadrine, quinine sulfate, tizanidine, and/or other muscle relaxants. The one or more NMDA receptor antagonists may include ketamine added to or included in the compounded transdermal cream comprising diclofenac, lidocaine, and prilocaine in an amount between approximately 0.1% and approximately 5.0% of the total weight of the final compounded transdermal cream.

In various embodiments, all or a portion of the one or more additional active ingredients may comprise a fine powder obtained by grinding up commercial tablets of the active ingredient. In some such embodiments or other embodiments, all or a portion of the one or more additional active ingredients may comprise a fine powder obtained from a bulk powder source. The one or more additional active ingredients may be dissolved or suspended in a solution or suspension or provided in a solution or suspension and subsequently combined with the lidocaine and prilocaine cream before, after, or along with, e.g., combined with, the diclofenac sodium solution.

In one particular embodiment, one or more additional active ingredients are provided in the form of a fine dry powder obtained from a bulk source or crushed commercial tablets, as described herein, may be dissolved or suspended in a diclofenac sodium 1.5% (w/w) or 2.0% (w/w) solution and then combined with the lidocaine 2.5% and prilocaine 2.5% cream to form the compounded transdermal cream for topical administration.

### IX. Additional Exemplary Embodiments

The present embodiments may include the presence of DMSO and/or Sterile Water for Irrigation, such as DMSO or Sterile Water for Irrigation in a sufficient quantity to allow for the topical delivery of the active ingredients mentioned herein. For instance, during the methods discussed herein, the DMSO may be removed and replaced with Sterile Water for Irrigation. The transdermal cream may be DMSO-free. The transdermal cream of the present embodiments may be compounded to have no bulk ingredients in it. For example, one or more of the ingredients may be obtained from crushing tablets comprising the ingredients. The tablets may comprise commercially available tablets formulated for oral administration. The tablets may therefore further include various excipients formulated for oral administration. According to some embodiments, the transdermal cream of the present embodiments may be compounded with one or more, including, in at least one embodiment, all ingredients obtained through bulk sources. The bulk sources may comprising one or more of the ingredients in a powder, such as a fine powder form.

In one aspect, compounded meloxicam, topiramate (and/or lamotrigine), lidocaine, and prilocaine cream may contain strictly commercially available medications. DMSO, which may be in some cream embodiments disclosed herein, may be replaced with Sterile Water for Irrigation. Sterile Water for Irrigation may act as a primary or sole penetration enhancer in some embodiments.

Although experimentation and investigation continues, it is believed that some detriments may develop from a transition to a DMSO-free compounded transdermal cream. It is believed that the removal of DMSO from certain compounds may decrease the effectiveness of the compound given that the primary penetrant is no longer present. Also, patients that have received the previous compounded version containing DMSO may experience lower efficacy rates. It is also believed that the transition of the formula may, at best, give the same efficacy that the patients previously had experienced, and, at worst, decrease efficacy due to the absence of DMSO.

On the other hand, the use of Sterile Water for Irrigation instead of DMSO may be cheaper and involve an easier method of manufacture. Also, Sterile Water for Irrigation is an FDA-approved commercially available medication.

In one embodiment, the transdermal cream comprises a nerve depressant, lidocaine, and prilocaine. A method of compounding the transdermal cream may comprise adding the fine powder medication comprising the nerve depressant to a starting transdermal cream or base. As described above, the fine powder medication may be obtained by crushing tablets of the medication, such as commercial tablets of the nerve depressant. As also described above, the fine powder medication may be obtained using bulk sources, which may include powder that may be ground to fine powder or the medication in a fine powder form. The fine powder of medication, e.g., nerve depressant, may be added to a transdermal cream or base composition containing both lidocaine and prilocaine. In another embodiment, the fine powder medication added to the transdermal cream or base composition further includes one or both lidocaine and prilocaine. In one particular instance of the above embodiment, the nerve depressant comprises or consists of gabapentin. In a further embodiment, gabapentin is added to the starting transdermal cream or base composition in a sufficient amount such that the final transdermal cream includes the approximately 1% to approximately 10%, approximately 3% to approximately 9%, or approximately 5% to approximately 8% by weight gabapentin and approximately 0.5% to approximately 5.0% by weight of each of lidocaine and prilocaine with DMSO or without DMSO. In one such embodiment gabapentin is added to the starting transdermal cream or base composition in a sufficient amount such that the final transdermal cream includes the approximately 6% by weight gabapentin and approximately 2% by weight of each of lidocaine and prilocaine. In one embodiment the final transdermal cream includes DMSO. In another embodiment the final transdermal cream does not include DMSO. For example, the method of compounding the above transdermal cream may include addition of DMSO to the starting transdermal cream or base or to the cream or base after gabapentin has been added or at an intermediate point of the compounding process. As described herein, all or a portion of the fine powder medication may be wetted with DMSO prior to addition to the transdermal cream. In embodiments without DMSO, the fine powder medication may be added directly to the cream or base or, in some embodiments, the fine powder medication may be wetted with liquid such as Sterile Water for Irrigation.

In a further embodiment, the transdermal cream comprises a nerve depressant, lidocaine, prilocaine, and a NSAID. The method of compounding the transdermal cream may further comprise adding the fine powder medication comprising the nerve depressant and NSAID to a starting transdermal cream or base. The fine powder medication comprising the fine powders of the nerve depressant and NSAID may be added together or separate. As described above, the fine powder medication may be obtained by crushing tablets of the medication, such as commercial tablets of the nerve depressant and the NSAID. As also described above, the fine powder medication may be obtained from bulk sources, which may include powder medication that may be ground to fine powder or the medication in a fine powder form. The fine powder medication, e.g., a nerve depressant and NSAID, may be added to the transdermal cream or base composition containing both lidocaine and prilocaine, which may be a commercially manufactured lidocaine and prilocaine cream, such as lidocaine 2.5% and prilocaine 2.5% cream. In another embodiment, the fine powder medication added to the transdermal cream or base composition further includes one or both lidocaine and prilocaine. In one particular instance of the above embodiment, the nerve depressant comprises or consists of gabapentin and the NSAID comprises or consists of diclofenac. In one embodiment, gabapentin and diclofenac are added to the starting transdermal cream or base composition in a sufficient amount such that the final transdermal cream includes approximately 1% to approximately 10%, approximately 1% to approximately 6%, or approximately 2% to approximately 5% by weight gabapentin, approximately 1% to approximately 10%, approximately 2% to approximately 8%, approximately 3% to approximately 7%, or approximately 4% to approximately 6% by weight diclofenac, and approximately 0.5% to approximately 5.0% by weight of each of lidocaine and prilocaine with DMSO or without DMSO. In one such embodiment gabapentin and diclofenac are added to the starting transdermal cream or base composition in sufficient amounts such that the final transdermal cream includes the approximately 3% by weight gabapentin, approximately 5% by weight diclofenac, and approximately 2% by weight of each of lidocaine and prilocaine. In one embodiment the final transdermal cream includes DMSO. In another embodiment the final transdermal cream does not include DMSO. For example, the method of compounding the above transdermal cream may include addition of DMSO to the starting transdermal cream or base or to the cream or base after gabapentin, diclofenac, or both have been added or at an intermediate point of the compounding process. As described herein, all or a portion of the fine powder medication may be wetted with DMSO prior to addition to the starting transdermal cream or base composition. In embodiments without DMSO, the fine powder medication may be added directly to the cream or base or, in some embodiments, the fine powder medication may be wetted with liquid such as Sterile Water for Irrigation.

In additional embodiments, the compounded transdermal cream comprises a nerve depressant, NSAID, lidocaine, prilocaine, and a muscle relaxant. The method of compounding the transdermal cream may further comprise adding the fine powder medication comprising the nerve depressant, NSAID, and muscle relaxant to a starting transdermal cream or base. The fine powder medication comprising the fine powders of the nerve depressant, NSAID, and muscle relaxant may be added together or separate. As described above, the fine powder medication may be obtained by crushing tablets of the medication, such as commercial tablets of the nerve depressant, the NSAID, the muscle relaxant. As also described above, the fine powder medication may be obtained from bulk sources, which may include powder medication that may be ground to fine powder or the medication in a fine powder form. The fine powder medication, e.g., the nerve depressant, the NSAID, and the muscle relaxant, may be added to the transdermal cream or base composition containing both lidocaine and prilocaine. In another embodiment, the fine powder medication added to the transdermal cream or base composition further includes one or both lidocaine and prilocaine, which may be a commercially manufactured lidocaine and prilocaine cream, such as lidocaine 2.5% and prilocaine 2.5% cream. In one particular form of the above embodiment, the nerve depressant comprises or consists of gabapentin, the NSAID comprises or consists of diclofenac, and the muscle relaxant comprises or consists of cyclobenzaprine. In one embodiment, gabapentin, diclofenac, and cyclobenzaprine are added to the starting transdermal cream or base composition in a sufficient amount such that the final transdermal cream includes the approximately 1% to approximately 10%, approximately 1% to approximately 6%, or approximately 2% to approximately 5% by weight gabapentin, approximately 1% to approximately 10%, approximately 2% to approximately 8%, approximately 3% to approximately 7%, or approximately 4% to approximately 6% by weight diclofenac, approximately 0.5% to approximately 1.5% by weight cyclobenzaprine and approximately 0.5% to approximately 5.0% by weight of each of lidocaine and prilocaine with DMSO or without DMSO. In one such embodiment gabapentin and diclofenac are added to the starting transdermal cream or base composition in sufficient amounts such that the final transdermal cream includes the approximately 3% by weight gabapentin, approximately 5% by weight diclofenac, approximately 1% by weight cyclobenzaprine, and approximately 2% by weight of each of lidocaine and prilocaine. In one embodiment the final transdermal cream includes DMSO. In another embodiment the final transdermal cream does not include DMSO. For example, the method of compounding the above transdermal cream may include addition of DMSO to the starting transdermal cream or base or to the cream or base after gabapentin, diclofenac, or both have been added or at an intermediate point of the compounding process. As described herein, all or a portion of the fine powder medication may be wetted with DMSO prior to addition to the starting transdermal cream or base composition. In embodiments without DMSO, the fine powder medication may be added directly to the cream or base or, in some embodiments, the fine powder medication may be wetted with liquid such as Sterile Water for Irrigation.

In one embodiment, the transdermal cream comprises a NSAID, lidocaine, and prilocaine. A method of compounding the transdermal cream may comprise adding the fine powder medication comprising the NSAID to a starting transdermal cream or base. As described above, the fine powder medication may be obtained by crushing tablets of the medication, such as commercial tablets of the NSAID. As also described above, the fine powder medication may be obtained using bulk sources, which may include powder that may be ground to fine powder or the medication in a fine powder form. The fine powder of medication, e.g., NSAID, may be added to a transdermal cream or base composition containing both lidocaine and prilocaine, which may be a commercially manufactured lidocaine and prilocaine cream, such as lidocaine 2.5% and prilocaine 2.5% cream. In another embodiment, the fine powder medication added to the transdermal cream or base composition further includes only one lidocaine and prilocaine. In one particular instance of the above embodiment, the NSAID comprises or consists of diclofenac. In a further embodiment, diclofenac is added to the starting transdermal cream or base composition in a sufficient amount such that the final transdermal cream includes the approximately 1% to approximately 10%, approximately 2% to approximately 8%, or approximately 4% to approximately 6% by weight of diclofenac and approximately 0.5% to approximately 5.0% by weight of each of lidocaine and prilocaine with DMSO or without DMSO. In one such embodiment either the diclofenac or the ibuprofen is added to the starting transdermal cream or base composition in a sufficient amount such that the final compounded transdermal cream includes the approximately 5% by weight diclofenac and approximately 2% by weight of each of lidocaine and prilocaine. In one embodiment the final compounded transdermal cream includes approximately 5% by weight diclofenac and DMSO. In another embodiment the final compounded transdermal cream does not include DMSO. For example, the method of compounding the above transdermal cream may include addition of DMSO to the starting transdermal cream or base or to the cream or base after diclofenac has been added or at an intermediate point of the compounding process. As described herein, all or a portion of the fine powder medication, for example, the diclofenac, may be wetted with DMSO prior to addition to the transdermal cream. In embodiments without DMSO, the fine powder medication, for example, the ibuprofen, may be added directly to the cream or base or, in some embodiments, the fine powder medication, for example, the ibuprofen, may be wetted with liquid such as Sterile Water for Irrigation.

In one aspect, a transdermal cream that permits the simultaneous administration of multiple medications in low concentrations may be provided. The transdermal cream may include lidocaine in an amount between approximately 0.5% and approximately 7.0% by weight of the transdermal cream; prilocaine in an amount between approximately 0.5% and approximately 7.0% by weight of the transdermal cream; meloxicam in an amount between approximately 0.01% and approximately 5.0% by weight of the transdermal cream; and lamotrigine in an amount between approximately 0.5% and approximately 5.0% by weight of the transdermal cream. In one embodiment, the transdermal cream may comprise approximately 2.0% by weight of both lidocaine and prilocaine, approximately 0.09% by weight meloxicam, and approximately 2.5% by weight lamotrigine. As a result, the transdermal cream may allow for the topical administration of lidocaine, prilocaine, meloxicam, and lamotrigine simultaneously during use. The transdermal cream may further include only or primarily Sterile Water for Irrigation as a penetration enhancer or other component, and be devoid of DMSO or DMSO-free.

In another aspect, a transdermal cream that permits the simultaneous administration of multiple medications in low concentrations may be provided. The transdermal cream may include lidocaine in an amount between approximately 0.5% and approximately 7.0% by weight of the transdermal cream; prilocaine in an amount between approximately 0.5% and approximately 7.0% by weight of the transdermal cream; meloxicam in an amount between approximately 0.01% and approximately 5.0% by weight of the transdermal cream; and topiramate in an amount between approximately 0.5% and approximately 5.0% by weight of the transdermal cream. In one embodiment, the transdermal cream may comprise approximately 2.0% by weight of both lidocaine and prilocaine, approximately 0.09% by weight meloxicam, and approximately 2.5% by weight topiramate. As a result, the transdermal cream may allow for the topical administration of lidocaine, prilocaine, meloxicam, and topiramate simultaneously during use. The transdermal cream may further include only or primarily Sterile Water for Irrigation for penetration enhancement or as a wetting component, and/or be devoid of DMSO or DMSO-free.

In another aspect, a method of compounding one or more medications with a transdermal cream for the topical administration of a compounded therapy may be provided. The method may include grinding up one or more tablets of a NSAID, an anticonvulsant, a nerve depressant, a muscle relaxant, a NMDA (N-Methyl-D-aspartate) receptor antagonist, an opiate or opioid agonist, and/or antidepressant into a fine powder of medication. In an alternate aspect, one or more, including all, of the medications may be obtained from bulk sources. The medications obtained from bulk sources may be in the form of a powder, which may be a fine powder or may be further ground into a fine powder prior to compounding with the transdermal cream or gel, which may be a commercially manufactured lidocaine and prilocaine cream, such as lidocaine 2.5% and prilocaine 2.5% cream. The method may include wetting the fine powder of medication mixture with DMSO or Sterile Water for Irrigation. The method may also include adding the fine powder of medication to a transdermal cream or base composition containing both lidocaine and prilocaine, the transdermal cream including both lidocaine and prilocaine in an amount of between approximately 0.5% and approximately 7.0% by weight of the transdermal cream, respectively. The method may include adding the fine powder of compounded medication to the starting transdermal cream or base composition in a sufficient amount such that the final transdermal cream includes the compounded medication that is ground up in a low amount of between approximately 0.01% and approximately 5.0% by weight of the transdermal cream. In one embodiment, an amount of ground up medication is added to the base composition such that the final transdermal cream contains low concentrations of several active ingredients and is approximately 2.0% by weight lidocaine, approximately 2.0% by weight prilocaine, approximately 0.09% by weight meloxicam, and approximately 2.5% by weight either lamotrigine or topiramate. In one embodiment, the transdermal cream may further include only or primarily Sterile Water for Irrigation for penetration enhancement or as a wetting component, and/or be devoid of DMSO or DMSO-free.

In another aspect, a method of compounding medications with a transdermal cream for the topical administration of a compounded therapy may be provided. The method may include grinding up tablets of two or more medications into a fine powder of compounded medication. The two or more compounded medications to be ground up may be selected from a NSAID, an anticonvulsant, a nerve depressant, a muscle relaxant, a NMDA receptor antagonist, a local anesthetic, an antidepressant, and an opioid or opiate agonist. The method may include wetting the fine powder of compounded medication with DMSO or Sterile Water for Irrigation. The method may include then adding the fine powder of compounded medication to a transdermal cream or gel such that the transdermal cream or gel allows for topical delivery of the two or more compounded medications for simultaneous treatment of two or more ailments when the transdermal cream or gel is topically applied. The transdermal cream may further include only or primarily Sterile Water for Irrigation for penetration enhancement or as a wetting component, and/or be devoid of DMSO or other penetration enhancers.

The present invention may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

What is claimed is:

1. A compounded transdermal cream comprising:
gabapentin, diclofenac, cyclobenzaprine, or a combination thereof;
lidocaine in an amount between approximately 0.5% to approximately 5.0% by weight of the compounded transdermal cream; and
prilocaine in an amount between approximately 0.5% to approximately 5.0% by weight of the compounded transdermal cream.

2. The compounded transdermal cream of claim 1, wherein gabapentin is present in an amount approximately 6% by weight of the compounded transdermal cream.

3. The compounded transdermal cream of claim 2, wherein lidocaine and prilocaine are each present in an amount approximately 2.0% by weight of the compounded transdermal cream.

4. The compounded transdermal cream of claim 3, wherein the compounded transdermal cream does not include DMSO.

5. The compounded transdermal cream of claim 1, wherein diclofenac is present in an amount approximately 5% by weight of the compounded transdermal cream.

6. The compounded transdermal cream of claim 5, further comprising DMSO, wherein lidocaine and prilocaine are each present in an amount approximately 2.0% by weight of the compounded transdermal cream.

7. The compounded transdermal cream of claim 6, wherein gabapentin is present in an amount approximately 3% by weight of the compounded transdermal cream.

8. The compounded transdermal cream of claim 7, wherein cyclobenzaprine is present in an amount approximately 1% by weight of the compounded transdermal cream.

9. The compounded transdermal cream of claim 5, wherein gabapentin is present in an amount approximately 3% by weight of the compounded transdermal cream, and wherein the compounded transdermal cream does not include DMSO.

10. The compounded transdermal cream of claim 9, wherein lidocaine and prilocaine are each present in an amount approximately 2.0% by weight of the compounded transdermal cream.

11. The compounded transdermal cream of claim 9, wherein cyclobenzaprine is present in an amount approximately 1% by weight of the compounded transdermal cream.

12. The compounded transdermal cream of claim 11, wherein lidocaine and prilocaine are each present in an amount approximately 2.0% by weight of the compounded transdermal cream.

13. A compounded transdermal cream comprising:
ibuprofen in an amount approximately 5% by weight of the compounded transdermal cream;
lidocaine in an amount between approximately 0.5% to approximately 5.0% by weight of the compounded transdermal cream; and
prilocaine in an amount between approximately 0.5% to approximately 5.0% by weight of the compounded transdermal cream,
wherein the compounded transdermal cream does not include DMSO.

14. The compounded transdermal cream of claim 13, wherein lidocaine and prilocaine are each present in an amount approximately 2.0% by weight of the compounded transdermal cream.
